(19) United States
(12) Patent Application Publication
(10) Pub. No.: US 2006/0247220 A1
(43) Pub. Date: Nov. 2, 2006

(54) Preventing the crystallization of cholesterol using oxysterols

(76) Inventor: Louis H. Krut, St. Louis, MO (US)

Correspondence Address: DAVIDSON BERQUIST JACKSON & GOWDEY LLP, 4300 WILSON BLVD., 7TH FLOOR, ARLINGTON, VA, 22203, US

(21) Appl. No.: 10/568,676

(22) PCT Filed: Aug. 19, 2004
(86) PCT No.: PCT/US04/26957

§ 371 Date: Jun. 21, 2006

Related U.S. Application Data
(60) Provisional application No. 60/496,641, filed on Aug. 20, 2003.

(51) Int. Cl. A61K 31/57; A61K 31/56; C07J 9/00

(52) U.S. Cl. 514/178; 552/540

(57) ABSTRACT

A therapeutic agent in the treatment of humans or animals wherein the agent is oxysterol, and a method of treatment wherein the agent is administered orally, parenterally, transdermally, buccally, sublingually or otherwise to deliver a sufficient amount of oxysterol to the human or animal to prevent or delay plaque formation in atherosclerosis by preventing or delaying the crystallization of cholesterol.

### CROSS REFERENCE TO RELATED APPLICATIONS

This application claims the benefit of U.S. Provisional Patent Application Ser. No. 60/496,641, filed Aug. 20, 2003, which is herein incorporated by reference and continued preservation of which is requested.

### THE FIELD OF THE INVENTION

The field of the invention relates to the prevention of plaque formation in atherosclerosis by preventing the crystallization of cholesterol. More specifically, this invention is directed to the use of autoxidation products of cholesterol (oxysterols) to prevent the crystallization of cholesterol, which may prevent or delay plaque formation in atherosclerosis.

### BACKGROUND OF THE INVENTION

Atherosclerosis is a leading cause of death in the United States. It is believed that the disease results from excess cholesterol from plasma accumulating in the arterial walls, which forms plaques that decrease blood flow and promote clot formation, ultimately causing heart attacks, stroke and claudication.

The process of plaque formation involves first the formation of lesions in the systemic arteries. There are three major classes of lipids that accumulate in lesions of atherosclerosis. The three are phospholipids, cholesterol, and cholesterol esters. These lipids are water insoluble. Phospholipids and cholesterol esters are almost mutually insoluble, but cholesterol, a crystalline solid at 37 degrees C., has considerable solubility in phospholipid bilayers and cholesterol esters. In normal infant intima, cholesterol is solubilized by phospholipid membranes. During fatty streak development, groups of cells are stimulated to take up more cholesterol than they excrete. The excess cholesterol is biochemically converted to cholesterol ester, which separates as droplets to form foam cells.

Some fatty streaks then undergo a transition to an intermediate lesion containing excess cholesterol which is carried in cholesterol-superstaturated membranes and droplets. Their formation coincide with the onset of necrosis and plaque formation. The hallmark of plaque is the presence of inert cholesterol crystals. They appear to form from hydrolysis of older deposits of cholesterol esters in the base of intermediate lesions. Thus lipids in plaque are stratified, with recently deposited cholesterol esters present in the luminal part of the intima and older deposits in the deeper regions.

Some prior art indicates that when plasma cholesterol is lowered below about 150 mg/dl, lipids are mobilized from lesions and regression gradually occurs. Early in the regression process, cholesterol esters are reduced at least partly by hydrolysis to yield cholesterol, some of which may crystallize and inhibit rapid regression. After prolonged periods of low plasma cholesterol, cholesterol esters and foam cells disappear and crystalline cholesterol gradually dissolves, leading to true regression.

Other art, however, contradicts that position. The cholesterol in the atherosclerotic plaque is contained in a gruel of “pultaceous necrotic debris”. It is not part of an active metabolic pool. Experimentally, when foam cell lesions are induced by feeding cholesterol to rabbits and the rabbits later returned to their normal diet, the plasma cholesterol rapidly fell to normal. When these animals were then evaluated for a year or two, there was no evidence that cholesterol cleared from the aorta, despite exceedingly low plasma cholesterol concentration. However, the lesions induced early in the experiment showed the development of progressive sclerosis, converting the fatty streaks into atherosclerotic plaques.

One view on oxysterols is that they are angiotoxic and atherogenic. This position seems to be attributable primarily to the work of one group reporting that oxysterols are more “angiotoxic” than cholesterol after gavage of a single dose of each in rabbits, and after repeated gavage of both cholesterol and oxysterol. Toxicity to aortic smooth muscle cells in culture was also noted. This work let to postulation that some oxysterols are probably the prime cause of atherosclerotic lesions and that the deposition of cholesterol and its esters is merely a secondary phenomenon.

Wilkens and Krut made a study on the effect of plasma lipids on the crystallization of cholesterol from a supersaturated solution in vitro. The results indicated that autoxidation products of cholesterol had a role in preventing the crystallization of cholesterol. Krut later demonstrated that small amounts of autoxidation products of cholesterol act synergistically with small amounts of phosphatidylcholine to prevent the crystallization of cholesterol in a supersaturated solution. In an in vivo study, Krut subcutaneously implanted tablets made of cholesterol in rats. These tablets were rapidly sequestered by fibrous tissue and no cholesterol was cleared. Tables made of cholesterol mixed with oxysterols were simultaneously subcutaneously implanted in the same rats, and these tables were rapidly brought into solution. The tablets cleared completely, leaving little fibrosis.

Schwenk et al. fed rabbits purified cholesterol, oxysterols alone, and oxysterols with cholesterol. It was found that the animals fed oxysterols alone had no atherosclerotic lesions, while the animals fed purified cholesterol showed both elevated serum cholesterol and lesions. Oxysterols fed together with cholesterol markedly attenuated lesion formation, despite elevated serum cholesterol levels.

Higley et al. fed rabbits purified cholesterol, oxysterols and a mixture of cholesterol and oxysterols. They found that cholesterol is much more atherogenic to rabbits than oxysterols or a mixture of cholesterol and oxysterols. A follow-up of Higley et al.'s study was made by Tipton et al. Partially purified cholesterol hydroperoxides, with or without pure cholesterol were fed to rabbits. It was found that cholesterol in the diet caused extensive atheroma formation in the aortas, but the addition of cholesterol hydroperoxides markedly reduced lesion formation, despite grossly elevated plasma cholesterol concentration in both groups.

None of the above references address the problem of crystallized cholesterol in plaque formation, or the proposed method of preventing the crystallization using oxysterols.

It is the purpose of the present invention to prevent the formation of cholesterol crystals which are present in plaque using a therapeutic amount of oxysterols. If the crystallization of cholesterol is prevented, plaque may not form, and atherosclerosis may be prevented.

### SUMMARY OF THE INVENTION

The present invention relates to the use of at least one oxysterol as a therapeutic agent in the treatment of humans or animals wherein oxysterol is administered orally, parenterally, transdermally, buccally, sublingually or otherwise to deliver a sufficient amount of oxysterol to the human or animal to prevent or delay plaque formation in atherosclerosis by preventing or delaying the crystallization of cholesterol. The present invention further involves a method of preventing or delaying plaque formation in atherosclerosis by administering a therapeutic amount of at least one oxysterol to a human or animal.

### DETAILED DESCRIPTION OF THE INVENTION

The present invention relates to the use of therapeutic amounts of at least one oxysterol to prevent the crystallization of cholesterol, thereby preventing or delaying plaque formation in atherosclerosis. Oxysterols includes a group of compounds that are formed by the oxidation of cholesterol. Oxysterols can be made by heating cholesterol in an oven with air. Oxysterols include those compounds found in source materials for cholesterol. Such source materials include sheep wool fat, which is rich in oxysterols, and beef spinal cord. Oxysterols are formed by the autoxidation of cholesterol and are included in this group. Further, cholesterol is known to autoxidize to form hydroperoxides. The term “oxysterols” includes such hydroperoxides. The term “oxysterols” includes individual compounds or mixtures of such compounds. In a preferred embodiment of the invention, the term “oxysterol” means a mixture of compounds.

A therapeutic amount of oxysterols may be administered orally. In a preferred embodiment, the oxysterol can be administered orally as a pill, tablet, bolus, gel capsule, liquid, suspension, solution, syrup, powder or mixtures thereof. The oxysterol embodied above can optionally include fillers, flavorings and sweeteners. Further, the oxysterol may be micro encapsulated.

Oxysterols can also be administered transdermally, in a cream or lotion, or in the form of an emulsion. Said cream or lotion is administered to the skin so as to be absorbed into the blood stream. In a preferred embodiment, the oxysterol is administered with lanolin, and the lotion or cream is applied to the skin. Further, transdermal patches can be used to administer the oxysterol. Other means of administering oxysterol are contemplated in this invention, including buccal, sublingual or parenteral administration.

### EXAMPLES OF THE INVENTION

The following examples illustrate the use of oxysterol to prevent the crystallization of cholesterol in lipids, which, in turn may prevent or delay plaque formation and atherosclerosis. These examples are not meant to limit the present invention in any manner.

Cholesterol was partially oxidized by heating in an oven with air at 80° C. Gas Chromatography/Mass Spectrometry analysis of this material showed numerous new oxysterol compounds generated, accounting for about 12% of the total sterols.

A 140 mg portion of the partially oxidized cholesterol was placed in a glass tube and 2.6 mL sunflower seed oil was added. The same amount of oil was added to tubes containing 60, 80, 100, 120 and 140 mg of pure cholesterol. All the tubes were held in a heating block at 95° C. until the sterols were dissolved. They were then held at room temperature (about 24° C.) for at least 3 weeks. The tubes containing 80 mg or more pure cholesterol formed a supersaturated solution on coating, and crystallization occurred promptly. The cholesterol in the tubes containing 80, 100, 120 and 140 mg pure cholesterol had completely crystallized in about 1 hour. The 60 mg sample was not a supersaturated solution, and crystallization did not occur. The partially oxidized cholesterol solution, which contained about 123 mg cholesterol and 17 mg oxysterols, remained clear for weeks with no crystallization.

To determine the maximum solubilizing effect of the oxysterols, 35 mg and 70 mg of pure cholesterol were added to tubes containing 140 mg of the partially oxidized cholesterol, and 2.6 mL oil was added to each tube. The mixtures were brought into solution as described above, and held at room temperature. There was no crystallization in the preparation containing the additional 35 mg of cholesterol. There was prompt crystallization in the preparation containing the additional 70 mg of cholesterol. Thus under these conditions, about 17 mg oxysterols can hold about 158 mg of cholesterol in solution in a lipid.

1. A therapeutic agent in the treatment of humans or animals to prevent or delay plaque formation and atherosclerosis by preventing or delaying the crystallization of cholesterol comprising at least one oxysterol.

2. The therapeutic agent of claim 1, wherein the at least one oxysterol is in a form suitable to be administered orally, parenterally, transdermally, buccally, sublingually or otherwise to deliver a sufficient amount of oxysterol to prevent or delay plaque formation in atherosclerosis.

3. The therapeutic agent of claim 1, wherein the at least one oxysterol is in a form suitable to be administered orally as a pill, tablet, bolus, gel capsule, liquid, suspension, solution, syrup, powder or mixture thereof.

4. The therapeutic agent of claim 1, wherein the at least one oxysterol is in a form suitable to be administered transdermally, in a cream or lotion, or in the form of an emulsion or a patch.

5. The therapeutic agent of claim 1, wherein the at least one oxysterol is a mixture of oxysterols formed from the oxidation of cholesterol.

6. A method of preventing or delaying plaque formation and atherosclerosis by preventing or delaying the crystallization of cholesterol by administering at least one oxysterol to a human or animal in an amount sufficient to prevent or delay plaque formation or atherosclerosis.

7. The method of claim 6, wherein the at least one oxysterol is administered orally, parenterally, transdermally, buccally, sublingually or otherwise to deliver a sufficient amount of oxysterol to prevent or delay plaque formation in atherosclerosis.

8. The method of claim 6, wherein the at least one oxysterol is administered orally as a pill, tablet, bolus, gel capsule, liquid, suspension, solution, syrup, powder or mixture thereof.

9. The method of claim 6, wherein the at least one oxysterol is administered transdermally, in a cream or lotion, or in the form of an emulsion or a patch.

10. The method of claim 6, wherein the at least one oxysterol is a mixture of oxysterols formed from the oxidation of cholesterol.
